(19) United States
(12) Patent Application Publication
(10) Pub. No.: US 2017/0354828 A1
(43) Pub. Date: Dec. 14, 2017

(54) AROMATIC POLYAMIDE FIBER MATERIAL SEPARATORS FOR USE IN ELECTROLYTIC CAPACITORS

(71) Applicant: Pacesetter, Inc., Sunnvyale, CA (US)

(72) Inventors: David R. Bowen, Taylors, SC (US); Ralph Jason Hemphill, Sunset, SC (US); Peter Fernstrom, Pickens, SC (US); Thomas F. Strange, Easley, SC (US); Kurt J. Erickson, Anderson, SC (US)

(21) Appl. No.: 15/623,251

(22) Filed: Jun. 14, 2017

Related U.S. Application Data
(60) Provisional application No. 62/349,790, filed on Jun. 14, 2016.

(51) Int. Cl. A61N 1/39; H01G 11/52; H01G 11/60; H01G 11/84; H01G 11/26

(52) U.S. Cl. CPC A61N 1/3956; H01G 11/84; H01G 11/26; H01G 11/60; H01G 11/52

(57) ABSTRACT

A capacitor includes an anode foil, a cathode foil, a conductive electrolyte, and a separator between the cathode foil and the anode foil. The conductive electrolyte fills between the cathode foil and the anode foil and contains butyrolactone. The separator includes an aromatic polyamide fiber material. The aromatic polyamide fiber material is non-woven and includes a para-aromatic-polyamide synthetic fiber. The separator has a thickness in a range of about 5 μm to about 20 μm and a density of greater than about 1.0 g/cm³.

### PRIORITY

This application claims the benefit of U.S. Provisional Application No. 62/349,790, entitled “AROMATIC POLYAMIDE FIBER MATERIAL SEPARATORS FOR USE IN ELECTROLYTIC CAPACITORS,” filed Jun. 14, 2016, which is incorporated herein by reference in its entirety to provide continuity of disclosure.

### FIELD

The present invention relates generally to the field of electrolytic capacitors, and more particularly, to separators for use in electrolytic capacitors.

### BACKGROUND

Compact, high voltage capacitors are utilized as energy storage devices in many applications, including implantable medical devices. These capacitors are required to have a compact design and to have high energy density, in order to deliver high voltage pulses when required. This is particularly true when the capacitors are used in an Implantable Cardioverter Defibrillator (ICD), also referred to as an implantable defibrillator, where high voltage electrolytic capacitors are used to deliver the defibrillation pulse and can occupy as much as one third of the ICD volume.

Implantable Cardioverter Defibrillators, such as those disclosed in U.S. Pat. No. 5,131,388, incorporated herein by reference, typically use two electrolytic capacitors in series to achieve the desired high voltage for shock delivery. For example, an implantable cardioverter defibrillator may utilize two 300 to 500 volt electrolytic capacitors in series to achieve a voltage of 600 to 1000 volts. A subcutaneous implantable cardioverter defibrillator (SICD) may utilize three or more 300 to 500 volt electrolytic capacitors in series to achieve a voltage of 900 volts to 1500 volts.

Electrolytic capacitors are used in ICDs, because they have the most nearly ideal properties in terms of size, reliability and ability to withstand relatively high voltage. Conventionally, such electrolytic capacitors include an etched aluminum foil anode, an aluminum foil or film cathode, and an interposed Kraft paper or fabric gauze separator impregnated with a solvent-based liquid electrolyte. While aluminum is the preferred metal for the anode plates, other metals such as tantalum, magnesium, titanium, niobium, zirconium and zinc may be used. A typical solvent-based liquid electrolyte may be a mixture of a weak acid and a salt of a weak acid, preferably a salt of the weak acid employed, in a polyhydroxy alcohol solvent. The electrolytic or ion-producing component of the electrolyte is the salt that is dissolved in the solvent. The entire laminate is rolled up into the form of a substantially cylindrical body, or wound roll, that is held together with adhesive tape and is encased, with the aid of suitable insulation, in an aluminum tube or canister. Connections to the anode and the cathode are made via tabs. Alternative flat constructions for aluminum electrolytic capacitors are also known, comprising a planar, layered, stack structure of electrode materials with separators interposed there between, such as those disclosed in the above-mentioned U.S. Pat. No. 5,131,388.

In ICDs, as in other applications where space is a critical design criterion, it is desirable to use capacitors with the greatest possible capacitance per unit volume. Since the capacitance of an aluminum electrolytic capacitor is provided by the anodes, a clear strategy for increasing the energy density in the capacitor is to minimize the volume taken up by paper and cathode and maximize the number of anodes. A multiple anode stack configuration requires fewer cathodes and paper separators than a single anode configuration and thus reduces the size of the device. A multiple anode stack consists of plurality of stacked units. Each stacked unit includes a cathode, a first paper separator, two or more anodes, a second paper separator, and a cathode. Neighboring stacked units can share the cathode between them, and a plurality of stacked units are placed within a capacitor case.

The separators serve to electrically insulate the cathode from the anodes to prevent an electrical short circuit there between. The material from which the separator is formed is selected to provide a desired voltage withstand (or breakdown voltage) of the capacitor for a desired thickness. Paper separators are commonly used in flat, stacked electrolytic capacitors, but paper is susceptible to dimensional instability due to water/humidity. The paper also is susceptible to thermal damage during the process of welding the capacitor case together. The safe upper thermal limit of the paper is on the order of 150° C. before damage occurs. Various manufacturing processes conventionally employed to assemble a capacitor can challenge that upper limit, causing reduced net manufacturing yield. In addition, during conventional manufacturing processes, the edges of the anodes may become rough and may contain burrs and particles that can protrude through the thin paper separators and electrically short the anode and cathode electrodes. When this happens, the capacitor's ability to store charge is compromised and the capacitor's lifetime is adversely affected.

### BRIEF SUMMARY

Presented herein are a flat, stacked electrolytic capacitor using an aromatic polyamide fiber material separator and methods for making same. Such separator materials not only electrically insulate the anodes and cathode electrodes but also withstand the environmental conditions described herein that are common in flat, stacked electrolytic capacitors.

According to an embodiment, a capacitor includes an anode foil, a cathode foil, and a separator between the cathode foil and the anode foil, wherein the separator comprises an aromatic polyamide fiber material. In an embodiment, the aromatic polyamide fiber material is non-woven and includes a para-aromatic-polyamide synthetic fiber. In one example embodiment, the separator has a thickness in a range of about 5 μm (microns) to about 20 μm and a density of greater than about 1.0 g/cm³. In another example embodiment, the separator has a thickness in a range of about 10 μm to about 20 μm and a density of between about 1.0 g/cm³ and about 1.2 g/cm³. In an embodiment, the separator is able to withstand a voltage of at least 460 V. In an embodiment, the separator is able to withstand a voltage of at least 600 V.

In an embodiment, the separator further includes cellulose. In an embodiment, the separator comprises a mixture of an aromatic polyamide fiber and a porous paper material.

In an embodiment, the separator comprises two or more discrete layers. In an embodiment, the separator comprises a porous paper material layer and an aromatic polyamide fiber layer. The first porous paper material layer may have a thickness, for example, in a range of about 5 μm to about 10 μm, and the aromatic polyamide fiber material layer may have a thickness, for example, in a range of about 5 μm to about 10 μm.

In an embodiment, the separator comprises two layers, each layer comprising a mixture of an aromatic polyamide fiber and a porous paper material, wherein the ratio of the aromatic polyamide fiber to porous paper material differs between the two layers.

The aromatic polyamide fiber material may be a sheet of non-woven aromatic polyamide fiber material, and the first porous paper material may be a sheet of Kraft paper.

According to one embodiment, the capacitor includes a plurality of anode foils electrically insulated from the cathode foil, wherein the plurality of anode foils, the cathode foil, and the separator form a first stacked unit. In another embodiment, the capacitor further includes a second stacked unit, wherein the second stacked unit includes at least one cathode foil, at least one anode foil, and at least one separator comprising an aromatic polyamide fiber material. The at least one cathode foil is insulated from the at least one anode foil by the at least one separator. In an embodiment, the stacked units are enclosed in a case, and the case is filled with an electrolyte.

According to one embodiment, the separator may comprise two aromatic polyamide fiber sheets joined together using a laser weld at a peripheral edge to form a sleeve such that the cathode foil is enclosed within the sleeve. The sleeve may further be sealed and/or adhered to a cathode tab of the cathode using a laser weld where the cathode tab exits the sleeve.

According to another embodiment, each separator further includes a first porous paper material adjacent to the aromatic polyamide fiber material. In an embodiment, the first porous paper material is laminated to the aromatic polyamide fiber material. In an embodiment, first porous paper material is calendered to the aromatic polyamide fiber material. In an embodiment, the aromatic polyamide fiber material is laminated and/or calendered to the porous paper material to form a composite separator such that the aromatic polyamide fiber material surrounds the outer perimeter of the porous paper material. In an embodiment, the composite separator is joined together with a second composite separator, also comprising aromatic polyamide fiber material surrounding an outer perimeter of a porous paper material, using a laser weld at the peripheral edge of the composite separator (the peripheral edge comprising the aromatic polyamide fiber material) to form a sleeve such that the cathode foil is enclosed within the sleeve. The sleeve may further be sealed and/or adhered to a cathode tab of the cathode using a laser weld where the cathode tab exits the sleeve.

The first porous paper material may have a thickness, for example, in a range of about 5 μm to about 10 μm, and the aromatic polyamide fiber material may have a thickness, for example, in a range of about 5 μm to about 10 μm. The aromatic polyamide fiber material may be a sheet of non-woven aromatic polyamide fiber material, and the first porous paper material may be a sheet of Kraft paper. The separator in this example embodiment may further include a second porous paper and a second aromatic polyamide fiber material, wherein the first porous paper and the second porous paper are arranged over both surfaces of the cathode foil, the first and second aromatic polyamide fiber material are arranged over both surfaces of the first and second porous paper, and the first and second aromatic polyamide fiber material are joined together at a peripheral edge to form a sleeve such that the first and second porous paper layers and the cathode foil are enclosed within the sleeve.

According to an embodiment, a method for forming a separator for a capacitor includes: providing an aromatic polyamide fiber material of a first thickness and a first density, and then calendering the aromatic polyamide fiber material down to a second thickness and a second density to form a calendered fiber material, wherein the first thickness is greater than the second thickness, and wherein the second density is greater than the first density. The first thickness may be, for example, in a range of about 30 μm to about 40 μm, and the second thickness may be, for example, in a range of about 10 μm to about 20 μm. The first density may be, for example, in a range of about 0.6 g/cm³ to about 0.8 g/cm³, and the second density may be, for example, in a range of about 1.0 g/cm³ to about 1.2 g/cm³. In an embodiment, the second density is greater than 1.0 g/cm³ and the second thickness is about 10 μm to about 20 μm.

In another embodiment, the method further includes, before calendaring the aromatic polyamide fiber material, adding a pulp into the aromatic polyamide fiber material such that the pulp is combined with the aromatic polyamide fiber material to form the calendered fiber material. In an embodiment, the pulp comprises cellulose, and a ratio of a volume of the aromatic polyamide fiber material to a volume of the pulp may be, for example, in a range of about 3:1 to about 1:1. In an embodiment, the composite material is calendered, such that the density of the composite separator is greater than 1.0 g/cm³ and the thickness is about 10 μm to about 20 μm. The calendered fiber material in this example can withstand a voltage of at least 600 V.

In an embodiment, the electrolyte used with capacitor includes butyrolactone. In an embodiment, the electrolyte used with capacitor includes a mix of butyrolactone and ethylene glycol to maintain a low ESR in the environment of a higher density separator and lack of (or lower concentration of) cellulose fibers.

In another embodiment, an implantable cardioverter defibrillator (ICD) or a subcutaneous implantable cardioverter defibrillator (SICD) comprising a capacitor is provided, the capacitor comprising: an anode foil, a cathode foil, a conductive electrolyte, wherein the conductive electrolyte fills between the cathode foil and the anode foil and contains butyrolactone, and a separator between the cathode foil and the anode foil, wherein the separator comprises an aromatic polyamide fiber material having a density of greater than about 1.0 g/cm³ and has a thickness in a range of about 5 μm to about 20 μm.

Further embodiments, features, and advantages of the present invention, as well as the structure and operation of the various embodiments of the present invention, are described in detail below with reference to the accompanying drawings.

### BRIEF DESCRIPTION OF THE DRAWINGS/FIGURES

The accompanying drawings, which are incorporated herein and form part of the specification, illustrate an electrolytic capacitor and a method for making same. Together with the detailed description, the drawings further serve to explain the principles of and to enable a person skilled in the relevant art(s) to make and use the devices and methods presented herein. In the drawings, like reference numbers indicate identical or functionally similar elements. Further, the drawing in which an element first appears is typically indicated by the leftmost digit(s) in the corresponding reference number.

FIG. 1 illustrates an electrolytic capacitor having a flat, stacked capacitor configuration according to exemplary embodiments of the present disclosure.

FIG. 2 illustrates a flowchart diagram of a method for manufacturing a separator according to exemplary embodiments of the present disclosure.

FIGS. 3A and 3D illustrate a cross section of one or more sleeves enclosing a cathode foil according to exemplary embodiments of the present disclosure.

FIGS. 3B, 3C, 3E, and 3F illustrate an exploded view of one or more sleeves enclosing a cathode foil according to exemplary embodiments of the present disclosure.

FIGS. 4A, 4B, and 4C illustrate a laser sealing process according to exemplary embodiments of the present disclosure.

FIGS. 5A, 5B, and 5C illustrate another laser sealing process according to exemplary embodiments of the present disclosure.

### DETAILED DESCRIPTION

The following detailed description refers to the accompanying drawings that illustrate exemplary embodiments. Other embodiments are possible, and modifications may be made to the embodiments within the spirit and scope of the disclosure presented herein. Therefore, the following detailed description is not meant to limit the scope of the disclosure. Rather, the scope of the invention is defined by the appended claims.

It would be apparent to a person skilled in the relevant art that the stacked electrolytic capacitor, as described herein, may be implemented in many different embodiments. Thus, the structure, operation, and method of making a capacitor are described with the understanding that modifications and variations of the embodiments are possible, given the level of detail presented herein. It will be apparent to a person skilled in the relevant art that the embodiments described herein may be used in a variety of devices and applications in addition to use in an implantable cardioverter defibrillator (ICD).

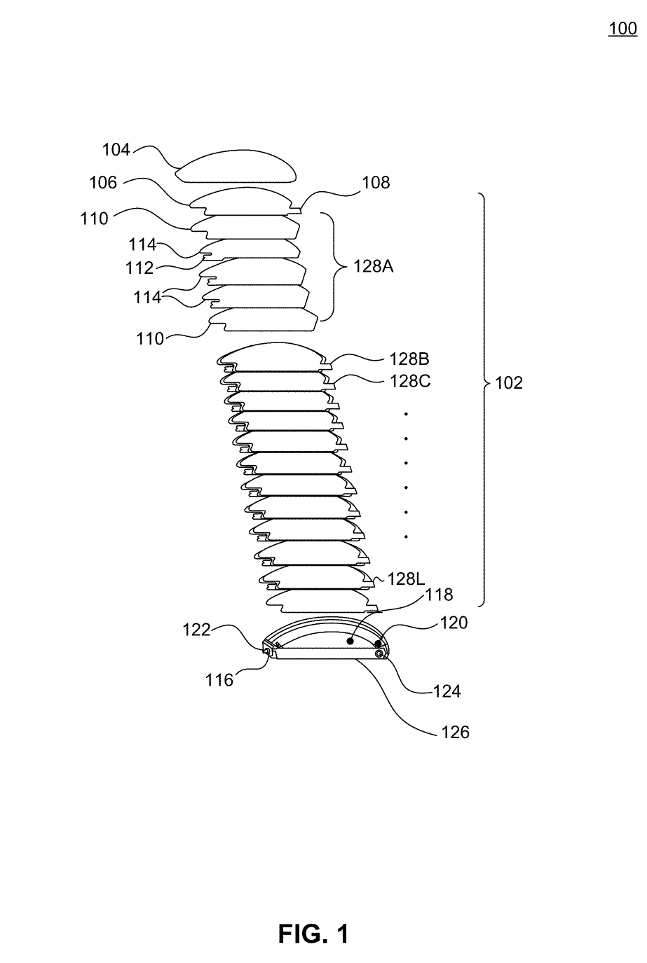

One aspect of the present disclosure provides an aromatic polyamide fiber material separator used in electrolytic capacitors. FIG. 1 illustrates an exemplary electrolytic capacitor 100 having a flat stack 102. Flat stack 102 includes at least one stacked unit 128A. Flat stack 102 can also include one or more additional stacked units, e.g., 128B-128L shown in FIG. 1. Each stacked unit can include the same or a different numbers of cathode foils 106 and anode foils 114. For illustrative purposes, parts in stacked unit 128A are described in detail herein.

Each of the stacked units 128 includes at least one cathode foil 106, at least one anode foil 114, and one or more separators 110 to electrically insulate the cathode foils 106 and the anode foils 114. In stacked unit 128A, anode foils 114 are stacked and a separator 110 is positioned between a cathode foil 106 and an adjacent anode foil 114, such that cathode foil 106 is electrically insulated from the adjacent anode foil(s) 114 by separator 110. Flat stack 102 is placed within a housing 126 enclosed by a lid 104. A conductive electrolyte fills the space between each of the elements within housing 126.

For illustration purposes, in FIG. 1, stacked unit 128A includes three metal foils or plates, representing anode foils 114, stacked together, e.g., on top of one another, and one cathode foil 106. In various embodiments, the stacked arrangement may have any reasonable number of anode foils 114 and cathode foils 106 per assembly or per stacked unit, depending on different design and/or application requirements. For example, a stacked unit 128 may include 2, 3, 4, 5, 6, 7, or 8 anode foils 114 and two or more cathode foils 106. Cathode foils 106 can be in any suitable arrangement. For example, a cathode foil 106 may be placed on top of the anode foils 114, below the anode foils 114, and/or between two anode foils 114. In another example, a cathode foil 106 may also be placed between two stacks of anode foils 114. A person skilled in the relevant art would understand how to determine the specific arrangement of the anode foils 114 and the cathode foils 106 according to different design or application requirements.

In the present disclosure, the terms “foil,” “sheet,” and “plate” are used interchangeably to refer to a thin, planar material. Cathode foils 106 are stacked on one or both sides of separators 110 to be electrically insulated from the anode foils 114. This arrangement can be repeated in a stacked manner to the appropriate thickness in order to give the necessary delivered energy/capacitance of the capacitor design.

Cathode foils 106 in stacked units 128 of flat stack 102 may be electrically connected to a single, common cathode terminal, and stacked anode foils 114 may be connected to a single, common anode terminal. In an embodiment, each of cathode foils 106 has a cathode tab 108 aligned with the cathode tabs 108 of adjacent cathode foils 106 so that cathode tabs 108 can be electrically connected (e.g., by welding) when the flat stack is assembled. Similarly, each anode foil 114 has an anode tab 112 aligned with the anode tabs 112 of adjacent anode foils 114 so that anode tabs 112 can be electrically connected (e.g., by welding) when flat stack 102 is assembled.

In an embodiment, housing 126 is an aluminum case that defines a chamber 118 in to which flat stack 102 is closely fit. Chamber 118 has a depth substantially equal to the thickness of flat stack 102. Housing 126 is provided with a feed through connector 116 through which an electrically conductive terminal or pin 122 extends. An insulating sleeve between terminal 122 and housing 126 forms an environmental seal. Anode tabs 112 are welded together and electrically connected to terminal 122 of feed through connector 116.

Housing 126 also includes a cathode attachment point (i.e., a location or area) 120 in the interior of the housing 126 at a position corresponding to cathode tabs 108. During assembly of capacitor 100, cathode tabs 108 are welded as a bundle to cathode attachment point 120 for electrical connection to housing 126.

In an embodiment in which cathode foils 106 are insulated from housing 126, in which housing 126 is non-conductive, or in which cathode foils 106 of different stacked units are insulated from one another, additional feed through connectors (not shown) may be used to provide electrical connection to each cathode through housing 126. Other suitable arrangements for housing 126 and connections to outside circuitry are possible without departing from the scope of the description above, as would be apparent to a person skilled in the relevant art.

In one embodiment, during assembly of capacitor 100, flat stack 102 (including one or more stacked units) is positioned in housing 126, anode tabs 112 are welded to feed through terminal 122, cathode tabs 108 are welded to housing 126, and lid 104 is sealed (e.g., via welding) to housing 126. Thereafter, housing 126 is filled with a conductive electrolyte (not shown) via a fill port 124. Fill port 124 is then sealed closed (e.g., via welding).

Exemplary materials for the plurality of cathode foils 106 include aluminum, titanium, stainless steel, and/or other high capacity, high surface area materials that work for a chosen electrolyte system. In an embodiment, titanium foils having a thickness of about 20 μm are used for cathode foils 106. Exemplary materials for the plurality of anode foils 114 include aluminum and tantalum. In an embodiment, aluminum foils having a thickness of between about 110 μm and about 115 μm are used for anode foils 114. Each anode foil 114 includes a dielectric material on or around its outer surface. The dielectric material may be a suitable oxide that is thermally grown on, or deposited onto, the surface of anode foil 114. A high-k dielectric material may be used for the dielectric material.

In an embodiment, in order to obtain higher capacitance, tunnels are etched through the thickness of anode foils 114, for example, by an electrochemical etching process. A widening process is then used to open the tunnels to prevent clogging during the later oxide formation step, for example, the tunnels may be widened using a solution of polystyrenesulfonic acid (PSSA). The PSSA improves the foil capacitance by protecting the foil surface from erosion and pitting. The tunnel widening procedure is described in more detail in co-owned U.S. Pat. Nos. 6,858,126, 6,802,954, or 8,535,507, incorporated herein by reference. Both the etch and widening processes can remove as much as 50% to 60% of the metal foil to create greater than 30 million tunnels per cm². An etched and formed anode foil 114 is punched by use of a mechanical die into an anode shape to conform to the desired geometry of housing 126. After anode foils 114 are punched by the mechanical die, they are assembled into stacks with cathodes 106 and separators 110, and enclosed within housing 126 by lid 104.

It should be understood that the various elements and dimensions of capacitor 100 are not drawn to scale. Although each of cathode foils 106, separators 110, and anode foils 114 are illustrated as being spaced apart from one another for the convenience of illustration and labeling, it would be understood by a person skilled in the relevant art that such elements are to be stacked together in close physical contact with one another.

Separators 110 are provided to maintain electrical insulation between each cathode foil 106 and the adjacent anode foils 114 within housing 126. Additionally, separators 110 are provided to prevent arcing between a cathode foil 106 and an adjacent anode foil 114 in spaces where the dielectric on the surface of the anode foil is very thin or nonexistent. Separators 110 are provided also to prevent arcing between a cathode foil 106 and an adjacent anode foil 114 where a void within electrolyte exists between a cathode foil 106 and an adjacent anode foil 114.

An aromatic polyamide fiber material is used for separator 110 between an anode foil 114 and an adjacent cathode foil 106. In an embodiment, the aromatic polyamide fiber material can be a suitable non-woven aromatic polyamide fiber material, for example, a membrane material made of non-woven aromatic polyamide fiber. In another embodiment, the aromatic polyamide fiber material can be a para-aromatic polyamide synthetic fiber material, such as a poly-paraphenylene terephthalamide (PPTA) fiber (trade name KEVLAR). The raw material of the aromatic polyamide fiber material may be provided as large sheets or on a roll, and may be cut (e.g., by punching or shearing) into the desired shape for separators 110. For example, a die punch may be used, or alternatively, the raw material may be laser cut.

In the present disclosure, the pore size, density, and fiber diameter of the aromatic polyamide fiber material are selected to give the desired electrical properties for operating under high voltage and low electrolyte conductivity. The selected pore size, density, and fiber diameter of the aromatic polyamide fiber material allow anode ions to pass through the pores and the aromatic polyamide fiber material to generate sufficiently high voltage. For example, in an embodiment, the aromatic polyamide fiber material is configured to withstand a voltage of above about 460 Volts and to have an absorbance of electrolyte to maintain a low ESR. If pores are too large, the voltage withstand is reduced.

In an embodiment, the aromatic polyamide fiber material has a density of at least about 1.0 g/cm³ to allow for a voltage withstand of at least about 600 Volts. In another embodiment able to withstand at least about 600 Volts, the aromatic polyamide fiber material has a thickness of about 10 μm to about 20 μm and a density of between about 1.0 g/cm³ to about 1.2 g/cm³, e.g., a density of about 1.06 g/cm³. In certain embodiments, the aromatic polyamide fiber material has a density greater than 1.0 g/cm³ and a thickness of about 10 μm to about 20 μm to allow for a voltage withstand of at least about 600 Volts. A density greater than 1.0 g/cm³ effectuates a more tortuous path through the separator, thereby increasing the standoff voltage, i.e., the break down voltage of the capacitor. Additionally, a more uniform density allows for use of a thinner and less dense material. Separators 110 should be sufficiently porous such that the electrolyte can penetrate through each separator 110 to allow ion exchange between anode foils 114 and cathode foils 106. If the pores are too small, the electrolyte may not properly impregnate separator 110. In certain embodiments, the pore size of the separator 110 is 0.05 μm to 5 μm. In certain embodiments, the pore size of the separator 110 is 0.1 μm to 2 μm.

By using a sufficiently strong aromatic polyamide fiber material for separators 110, the thickness of a separator containing aromatic polyamide fiber material can be reduced significantly, e.g., below the thickness of a conventional 20 μm-thick paper separator. Meanwhile, a separator 110 containing aromatic polyamide fiber material can have a higher puncture resistance, i.e., higher strength, than a conventional 20 μm-porous paper sheet. Such aromatic polyamide fiber material may have a fiber diameter not greater than (i.e., less than or equal to) about 10% of the thickness of the aromatic polyamide fiber material. For example, for a 20 μm-thick aromatic polyamide fiber material, the fiber diameter may be less than about 2 μm (e.g., about 0.1 μm to about 2 μm). In an embodiment, electrospun PPTA fibers on the order of about 1 μm in diameter may be used for separators 110 to achieve a thickness of between about 5 μm to about 20 μm. The allowance of a thinner separator 110 due to the stronger aromatic polyamide fiber material allows for improved packaging efficiency and enables more anode foils per given thickness. Therefore, a higher energy density can be realized for the capacitor. Unlike conventional paper separators, the disclosed separators 110 made of aromatic polyamide fiber material are less adversely affected by moisture and are more forgiving of mechanical stress. In addition, the aromatic polyamide fiber material can safely withstand much higher incidental thermal stress, on the order of about 400° C.-500° C., without detriment. These characteristics result in vastly larger windows of safety in manufacturing compared to paper separators.

As a result of the etching, widening and forming processes, an anode foil 114 becomes very brittle. The more metal removed (resulting in higher surface area), the harder the anode foil is to punch without forming cracks and particles. As a result of these manufacturing processes, the edges of an anode foil 114 after the manufacturing processes can contain burrs and attached particles. Separator breach from such particles and burrs on the edges of an anode foil 114 can be prevented by the separators made of aromatic polyamide fiber material as disclosed herein. Accordingly, the occurrence of short circuits between cathode foils 106 and anode foils 114 can be reduced, and the overall quality and lifetime of the capacitor can be improved. Hi-pot tests (a “high potential” or “hi-pot” test is also known as a “dielectric withstand test”) are performed to check for shorts between the stacked anode foils and the corresponding cathode foils before assembly. With conventional paper separators, failures can be as much as 5% to 10% depending on the brittleness of the anode foils. Use of aromatic polyamide fiber material reduces these yield losses significantly, nearly eliminating such failures.

In an embodiment, the aromatic polyamide fiber materials described herein may be combined with porous paper sheets to form a separator 110. For example, an aromatic polyamide fiber material having a thickness of about 10 μm may be stacked with a porous paper sheet having a thickness of about 10 μm to form a double-layered separator 110. The doubled layered separator 110 may have increased wettability and voltage withstand and improved durability as compared to a conventional 20 μm-thick paper separator.

In certain embodiments, an aromatic polyamide fiber sheet having a thickness of greater than 10 μm may be stacked with a porous paper sheet having a thickness of greater 10 μm and the two sheets may be calendered together in a damp state to form a double-layered separator 110, wherein the thickness of the composite double-layered separator 110 is 20 μm or less. The calendering process also advantageously increases the density of the composite double-layered separator 110, to a density of greater than about 1.0 g/cm³, thereby increasing the standoff voltage of the composite double-layered separator 110. The aromatic polyamide fiber sheet acts as “armor” for the porous paper sheet, advantageously providing a double-layered separator 110 that is less adversely affected by moisture, more forgiving of mechanical stress, and able to safely withstand much higher incidental thermal stress without detriment.

In an embodiment, the composite double-layered separator 110 comprises a porous paper sheet comprising Kraft paper having a thickness of between about 5 and about 10 μm and the aromatic polyamide fiber material sheet having a thickness of between about 5 and about 10 μm, so that the total combined thickness of the porous paper sheets and the aromatic polyamide fiber material sheet is not greater than (i.e., less than or equal to) about 20 μm. A plurality of alternating enclosed cathode foils and stacked anode foils, insulated by the separators containing aromatic polyamide fiber material sheets and porous paper sheets, can be stacked and/or calendered together to form the electrolytic capacitor. Kraft paper is commonly used as a separator in capacitors as would be understood by a person skilled in the relevant art. In various embodiments, the porous paper sheet can also be other kinds of porous, strong, electrically insulating, and durable paper sheets.

In yet other embodiments, separator 110 can contain an aromatic polyamide fiber material mixed with at least one other electrically-insulating material, such as a polymeric material, or cellulose. The polymeric material can include synthetic polymers, such as nylon, polyvinyl chloride, silicone, and polystyrene. The aromatic polyamide fiber material can form a stable bond with the other material such that the formed separator 110 has the desired characteristics of thickness, density, permeability, thermal stability, dimensional stability, and/or heat durability. In one embodiment, the separator 110 contains an aromatic polyamide fiber material mixed with cellulose.

In other embodiments, a separator 110 containing an aromatic polyamide fiber material mixed with at least one other electrically-insulating material (e.g., cellulose), as described above, is combined with a porous paper sheet, as described above.

In certain embodiments, a separator 110 containing an aromatic polyamide fiber material mixed with cellulose and/or porous paper is provided. The separator 110 may be manufactured by ultrasonic mixing, or high sheared mixing, of a cellulose pulp with an aromatic polyamide fiber, using a mix time of 5 minutes to one hour. In certain embodiments, a tertiary fiber, e.g., a low melting solid, such as polyvinyl alcohol, or a low melting wax, is mixed together with the cellulose pulp and aromatic polyamide fiber. In a second step, the paper/aromatic polyamide fiber mix is pressed and heated, in order to dissolve out the tertiary fiber, leaving a composite separator of a sufficient pore size such that the electrolyte may properly impregnate the separator, e.g., about 0.05 μm to about 5 μm.

A composite separator 110 containing an aromatic polyamide fiber material mixed with cellulose and/or porous paper may also be made using the methods described in U.S. Application Publication No. 2016-0293338 of U.S. patent application Ser. No. 15/184,157, filed Jun. 16, 2016, which is incorporated herein by reference, and/or by modifying the methods described therein with the present disclosure. In certain embodiments, an aromatic polyamide fiber blended cellulose/N-methylmorpholine N-oxide (“NMMO”) solution may be obtained by using total chlorine-free dissolving pulp having a degree of polymerization of 700 with a cellulose/NMMO ratio of 5/95, and adding sufficient aromatic polyamide fiber (e.g., aramid) having a diameter less than about 2 μm (e.g., about 0.1 μm to about 2 μm), so that the ratio of the volume of the aromatic polyamide fiber material to the volume of the pulp absorbed by the aromatic polyamide fiber material is about 3:1 to about 1:1. 5% by weight polyacrylamide, used as a flocculant, with respect to cellulose may be added as an active element to the obtained cellulose/NMMO solution. The solution may then be extruded through a 0.60 mm-wide slit using a T die-type extruder, passed through 10 mm air gaps, and then immersed in a coagulation bath of a 20% by weight NMMO poor solvent so as to regenerate the cellulose. Once the regenerated cellulose is washed in three washing baths of ion-exchanged water, the solvent is exchanged in three IPA baths and the composite material is dried in a drum-type dryer. In certain embodiments, the composite material may be supercalendered until the separator 110 has a thickness of about 10 μm to about 20 μm and a density greater than 1.0 g/cm³ and an average pore size of between about 0.05 μm to about 5 μm.

In certain embodiments, in addition to using a separator 110 consisting essentially of an aromatic polyamide fiber and/or a separator 110 fabricated using a ratio of the volume of the aromatic polyamide fiber material to the volume of the pulp absorbed by the aromatic polyamide fiber material of about 3:1 to about 1:1, a separator 110 comprising a mixture of cellulose/aromatic polyamide fiber created using a larger cellulose/aromatic polyamide fiber ratio may be used. According to an embodiment, an aramid fiber blended cellulose/NMMO solution may be obtained by using total chlorine-free dissolving pulp having a degree of polymerization of 700 with a cellulose/NMMO ratio of 5/95, and adding aramid fibers having an 8 μm diameter and 2 mm fiber length such that the cellulose/aramid fiber rate is 70/30 so as to dissolve the cellulose. 5% by weight polyacrylamide with respect to cellulose may be added as an active element to the obtained cellulose/NMMO solution. The composite material may then be extruded through a 0.60 mm-wide slit using a T die-type extruder, passed through 10 mm air gaps, and then immersed in a coagulation bath of a 20% by weight NMMO poor solvent so as to regenerate the cellulose. Once the regenerated cellulose is washed in three washing baths of ion-exchanged water, the solvent may be exchanged in three IPA baths and the cellulose may be dried in a drum-type dryer, thereby making a porous film having a thickness of 61.4 μm, density of 0.57 g/cm³, and an average pore size of 0.38 μm. In certain embodiments, the composite material may be supercalendered until the separator 110 has a thickness of about 10 μm. A layer having a higher concentration of aramid can be heat sealed and/or calendered with the layer having a lower concentration of aramid, to form a double-layered separator.

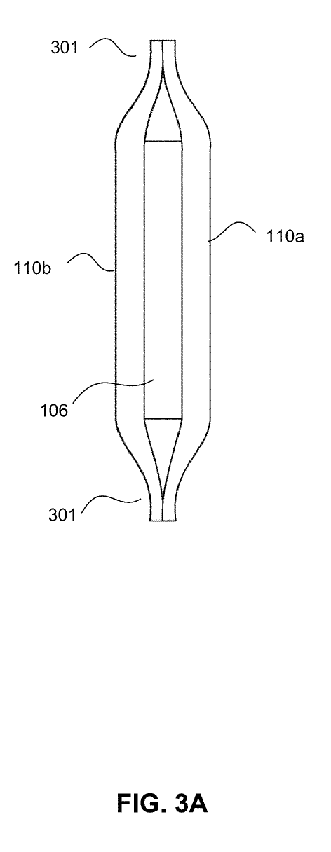

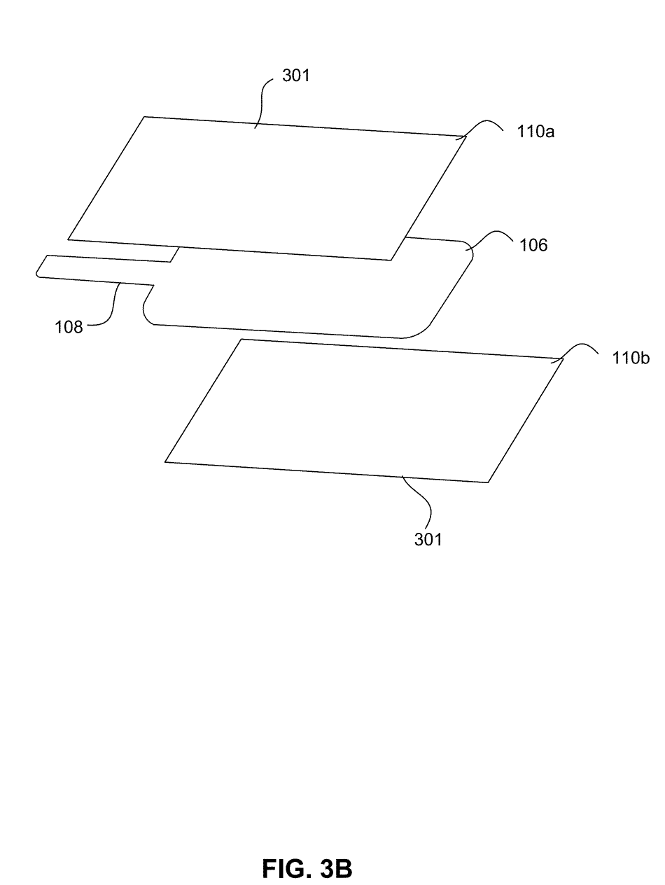

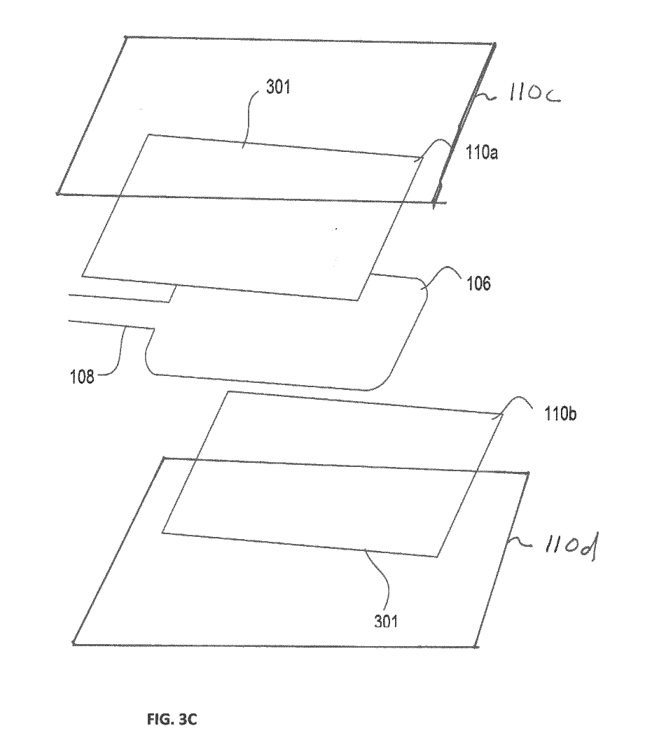

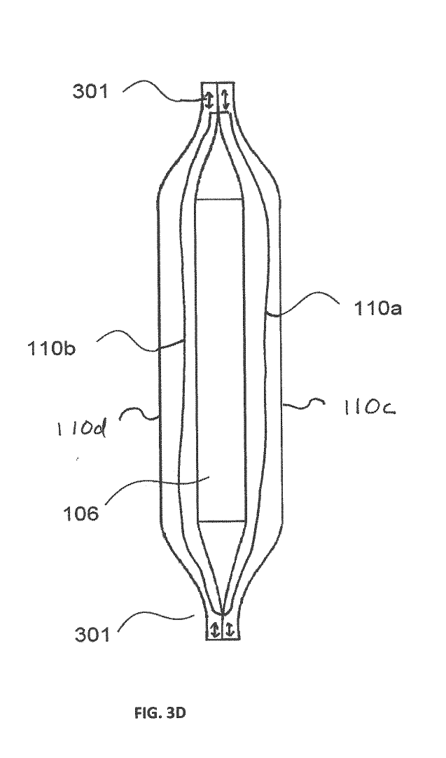

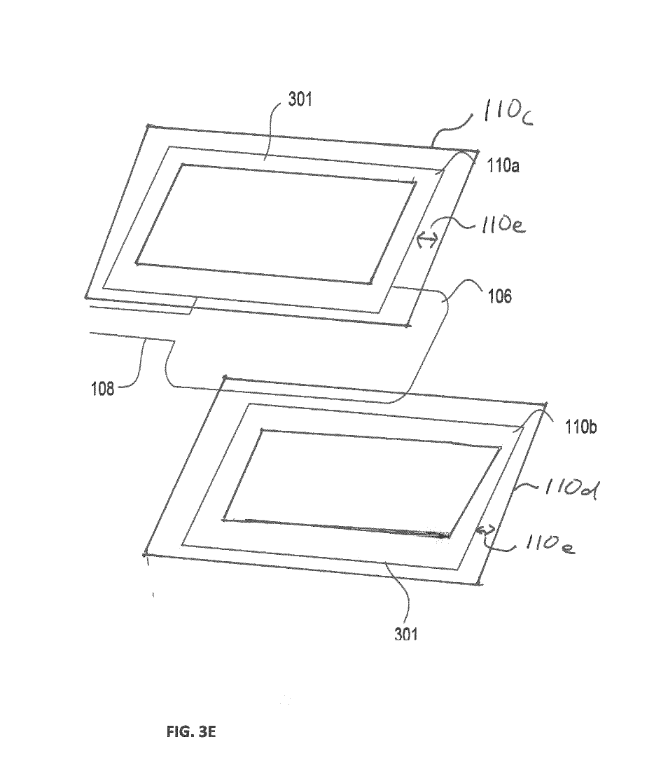

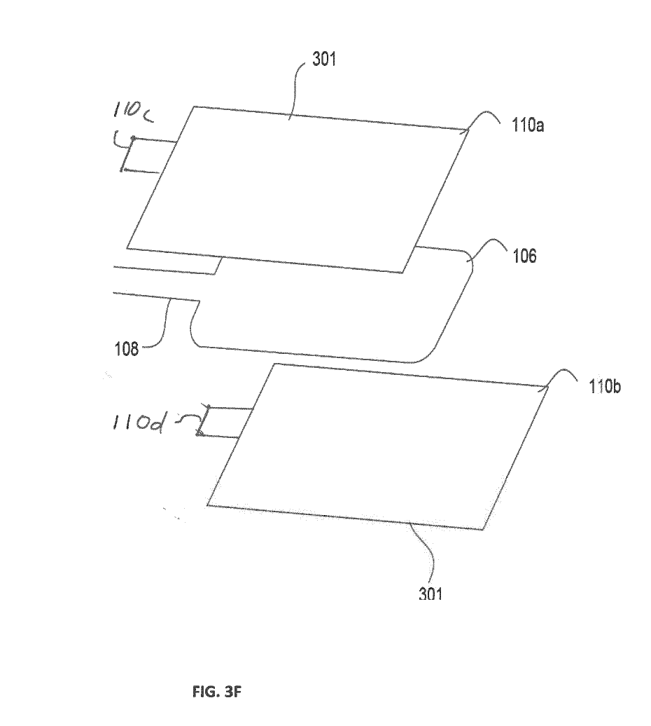

In an embodiment, two separators 110 may be sealed together at a peripheral edge to form a sleeve as is described in detail in co-owned U.S. Application Publication No. 2017-0110255 of U.S. patent application Ser. No. 14/882,782, filed Oct. 14, 2015, which is incorporated herein by reference. In this embodiment, as shown in FIGS. 3A-3E, cathode foil 106 is sandwiched or enclosed within a sleeve formed by the separators 110a and 110b. In certain embodiments, wherein the two porous paper separators form a paper sleeve or where the content of an aromatic polyamide fiber/paper composite separator comprises a proportion of paper to aromatic polyamide fiber that would subject the separator to significant thermal damage during a welding process, suitable adhesive can be used to seal the peripheral edge of the porous paper separators.

In embodiments, wherein two or more separators 110 consists essentially of an aromatic polyamide fiber, e.g., aramid, the peripheral or circumferential edges, shown as locations 301 in FIG. 3A, of separators 110 can advantageously be sealed together using a laser to melt the separator material together to form a continuous bond around cathode foil 106 up to cathode tab 108. A different laser parameter set can then be used, if desired, to advantageously laser bond separator 110 to cathode tab 108 to complete the seal around cathode foil 106.

The inventors believe that, laser sealing has certain advantages over adhesives to make the sleeve. For example, using a laser to heat seal allows for very accurate and precise application in very small areas and in very complex shapes with only a program change (rapid prototyping). The polymer properties of the aromatic polyamide fiber material allow a laser to heat seal the edges and heat seal to the cathode tab 108. The smaller the heat seal area, the more area the chemical/electrical properties of the separator 110 are maintained to provide the proper ESR. Additionally, the laser heat seal does not add significant thickness to the separator-cathode-separator at the cathode tab 108. In contrast, use of an adhesive would add a film thickness layer. The laser output parameters can be changed during an automatic sealing process, depending on the sealing area. The process of sealing separator to separator, and separator to cathode tab, can be one process. Further, laser adhesion does not suffer from potential chemical incompatibility of an adhesive to the electrolyte, which can cause problems over time. For example, an adhesive could react with the electrolyte and lose the strength of the seal and/or add impurities to the conductive electrolyte.

In certain embodiments, separators 110 comprising a mixture of an aromatic polyamide fiber, e.g., aramid and cellulose pulp, may also be sealed together using a laser to melt the separator material together to form a continuous bond around cathode foil 106 up to cathode tab 108, so long as the amount of cellulose break down during the welding process is not so great as to cause a malfunction.

In an embodiment, in forming a sleeve from the aromatic polyamide fiber material, a laser (e.g., a CO₂ or Nd:YAG laser, primarily operating in a continuous-wave mode, with respective wavelengths in the range of 10,600 nm or 1030 nm) can be used to cut the material to form two sheets that could then be joined or sealed together at a peripheral or circumferential edge. Alternatively, where thermal damage may be a concern or where non-homogenous behavior is observed from the aromatic polyamide fiber material that may provide unequal processing, a short-pulsed laser in the nanosecond range (also in the 1030 nm range) may bring some advantages. Using short pulses, multiple passes could be used to remove material while limiting heat input to the material. These same laser may be used to join or seal the edges of the sheets together.

FIG. 3C illustrate an embodiment where a top separator 110 includes an aromatic polyamide fiber material layer 110c and a porous paper separator 110a and a bottom separator 110 includes an aromatic polyamide fiber material layer 110d and a porous paper layer 110b. One or more porous paper layers 110a can be positioned between the cathode foil 106 and the aromatic polyamide fiber layer 110c, or between the aromatic polyamide fiber 110c and the adjacent anode foils 114. One or more porous paper layer 110b can be positioned between the cathode foil 106 and the aromatic polyamide fiber layer 110d. Porous paper layers 110a and 110b may be sealed together at a peripheral edge to form a sleeve as is described in detail in co-owned U.S. Application Publication No. 2017-0110255 of U.S. patent application Ser. No. 14/882,782, filed Oct. 14, 2015. Alternatively, porous paper layers 110a and 110b may not be sealed together by an adhesive (which adds bulk to the capacitor and thus the device, e.g., ICD or SICD) but may rely instead on the seal provided by laser boding of aramid layers 110c and 110d, as described in further detail herein.

As illustrated in FIGS. 3C and 3D, the aromatic polyamide fiber material 110c surrounds the outer perimeter of the porous paper material 110a. In certain embodiments, an aromatic polyamide fiber layer 110c and porous paper layer 110a are calendered together, such that a resulting composite double-layered separator comprises an aromatic polyamide fiber layer 110c (e.g., on the top) having a first width and first length and a porous paper layer 110a (e.g., on the bottom) having a second width and second length, wherein the first width and first length of the aromatic polyamide fiber layer 110c are larger than the second width and second length of the porous paper layer 110a. The distance 301 from an edge of the aromatic polyamide fiber material 110c and the outer peripheral edge of the porous paper material 110a may be sufficient to permit a weld to thermally bond the composite double-layered separator (comprising layers 110a and 110c) to another composite double-layered separator (comprising layers 110b and 110d) at only the aromatic polyamide fiber material (110c and 110d) to form a sleeve, without thermally damaging the paper separator layers (110a and 110b) because the laser may be directed outside the perimeter of the porous paper film. In certain embodiments, the distance 301 from an edge of the aromatic polyamide fiber material and an edge of the porous paper material is between 15 thousandths on an inch and 30 thousands of an inch. The aramid sleeve may further be sealed and/or adhered to a cathode tab of the cathode using a laser weld where the cathode tab exits the sleeve.

As illustrated in FIG. 3E, in certain embodiments, the aromatic polyamide fiber material 110c may be calendered, laminated, or otherwise sealed to the porous paper material 110a so as to form only a frame around the periphery of the porous paper film, such as that the composite sleeve can be laser sealed using the aromatic polyamide fiber material 110c and 110d outside the perimeter of the porous paper film at area 110e and/or to a cathode tab of the cathode where the cathode tab exits the sleeve (where there is only aromatic polyamide fiber material).

As illustrated in FIG. 3F, in certain embodiments, the aromatic polyamide fiber materials 110c and 110d may be calendered, laminated, or otherwise sealed to the porous paper separators 110a and 110b only in the area where the cathode tab 108 exits the sleeve to allow the sleeve to be laser welded to the cathode tab without thermally damaging the porous paper separators 110a and 110b.

In an embodiment, the composite paper/aromatic polyamide fiber separator is joined together with a second composite separator, also comprising aromatic polyamide fiber material surrounding an outer perimeter of a porous paper material, using a laser weld at the peripheral edge of the composite separator to form a sleeve such that the cathode foil is enclosed within the sleeve. The sleeve may further be sealed and/or adhered to a cathode tab of the cathode using a laser weld where the cathode tab exits the sleeve.

In another embodiment, the one or more porous paper separators can form a sleeve and be positioned between the cathode foil 106 and the aramid sleeve (i.e., a sleeve within a sleeve). In yet another embodiment, the one or more porous paper separators can form a sleeve to enclose the aramid sleeve containing the cathode foil (i.e., an aramid sleeve within a paper sleeve). In the present disclosure, terms “aramid” and “aromatic polyamide” are interchangeable and refer to the same material.

Optionally, one or more aromatic polyamide fiber material separators can be positioned between the cathode foil 106 and the paper sleeve, or between the paper sleeve and the adjacent anode foils 114. Positioning an aromatic polyamide fiber material separator/aromatic polyamide fiber material separator layer between a paper separator/paper separator layer and the anode foil (or encapsulating a cathode with a paper sleeve and encapsulating the paper sleeve with an aramid sleeve) advantageously places the aramid in a position where it can best defend against short circuits. In this position, the aramid may prevent burrs on the edges of the anodes and other particles from protrude through the thin paper separators.

It will be apparent to those skilled in the art that the number of sleeves, the materials and methods to form the sleeves are subjected to different application and design requirements. In various embodiments, by sealing the cathode tab 108 and the separators 110 together using a weld, a complete pocket is formed around the cathode foil 106, preventing particles formed from die cutting of anode foils 114/cathode foils 106, from penetrating the separators 110 or causing a short between the cathode foil 106 and anode foils 114. That is, aromatic polyamide fiber material separators 110 can reduce particle penetration and reduce shorts between cathode foil 106 and anode foils 114. In some embodiments, an aramid sleeve provides protection for a paper sleeve such that the paper sleeve is less susceptible to punctures caused by rough particles in the conductive electrolyte. Depending on the applications, in some embodiments, the aromatic polyamide fiber material separators forming the aramid sleeve may contain a suitable pulp, e.g., cellulose, as described herein.

To complete sealing of the aramid sleeve or the paper sleeve around cathode foil 106, the respective sleeve may be bonded to cathode tab 108.

According to an embodiment the aromatic polyamide fiber material is bonded to the cathode tab using a laser. Joining a polymer to a metal cannot normally be done with a fusion welding process, because the heat needed to melt the metal of the tab is well above the evaporation point of the polymer. And the heat needed to melt the polymer is well below any meaningful temperature for metal joining. In accordance with embodiments disclosed herein, however, the joining/sealing is facilitated by the addition of texturing or macrostructures formed on the tab. The texturing or macrostructures provide features for the aromatic polyamide fiber material to move into when the material is heated to or near its melting point with an appropriate laser source. For the aromatic polyamide fiber material according to some embodiments, a laser in the 1070-960 nm wavelength is suitable. The laser energy can then be transmitted through the aromatic polyamide fiber material, with the aromatic polyamide fiber material acting as a transmissive or partially transmissive layer. The laser energy then heats the metal of the cathode tab, melting the aromatic polyamide fiber material, and causing it to flow into the texture surface or macrostructure on the cathode tab. This creates interlocking features and approximates the joint geometry that could be obtained for materials that have a more similar melting temperature. A fixture may be used to hold the aromatic polyamide fiber material in contact with the cathode tab to obtain sufficient heat transfer. Each side of the tab can be joined to the aromatic polyamide fiber material sleeve individually, or simultaneously.

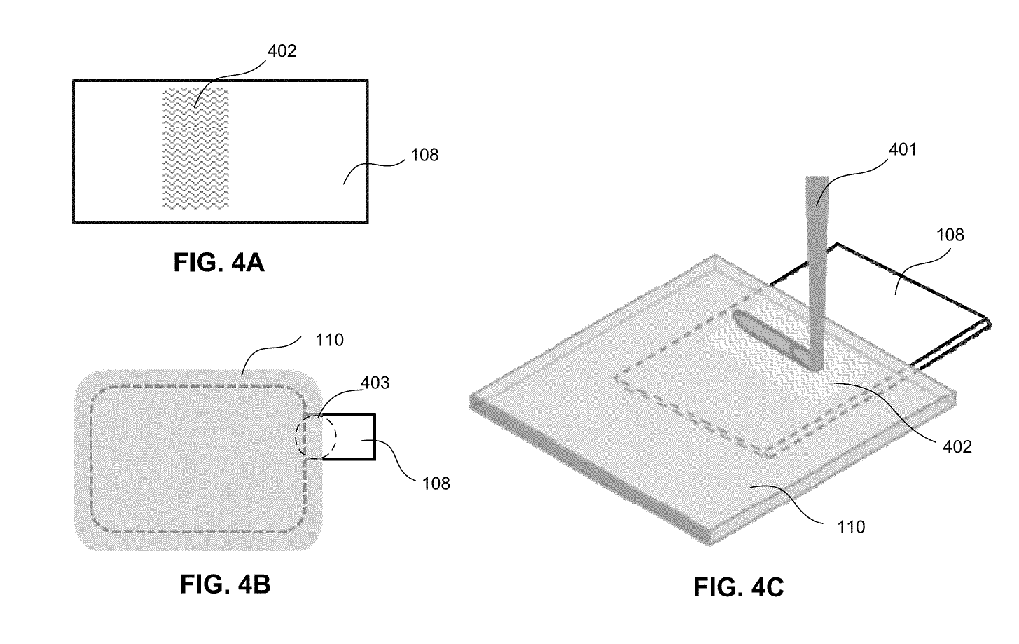

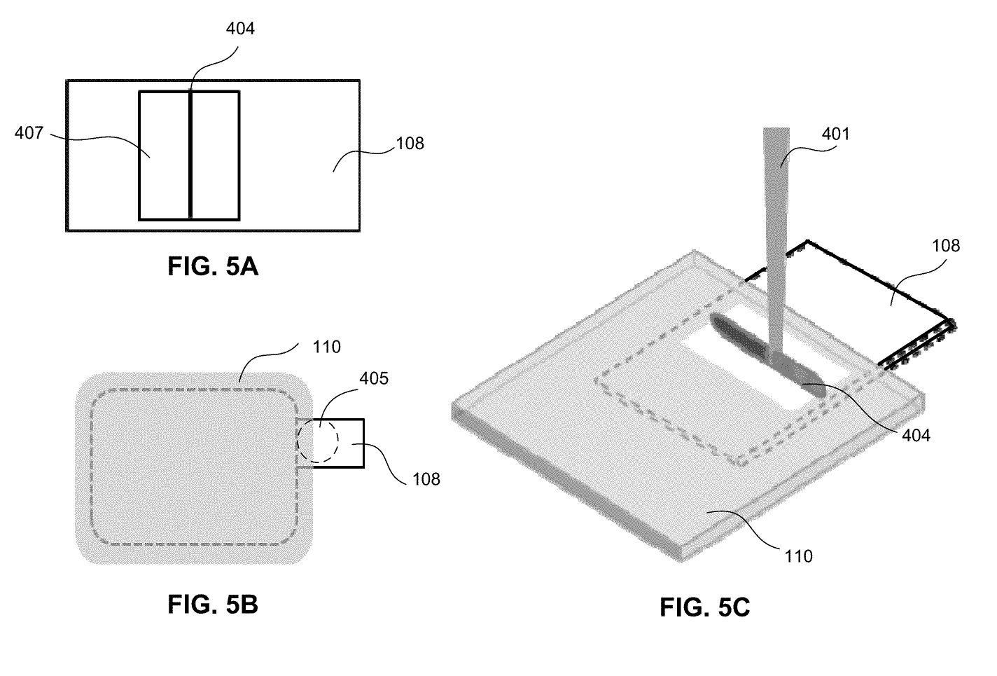

FIGS. 4A, 4B, and 4C, and FIGS. 5A, 5B, and 5C illustrate two processes of laser sealing. As shown in FIG. 4A, a disrupted or roughened surface 402 may be created on cathode tab 108. Disrupted surface 402 provides features for the aromatic polyamide fiber material separator 110 to move into when heated to or near its melting point. Bonding strength can thus be improved. To accomplish this bonding, separator 110 may be placed over cathode tab 108.

FIG. 4B illustrates a separator 110 positioned over a cathode tab 108, and an enlarged view of portion 403 is shown in FIG. 4C to illustrate a laser bonding process. As shown in FIG. 4C, laser source 401, provides laser energy, to a portion of separator 110 positioned adjacent disrupted surface 402, with the aromatic polyamide fiber material separator 110 acting as a transmissive or partially transmissive layer. The laser energy melts the aromatic polyamide fiber material and welds the aromatic polyamide fiber material separator 110 onto cathode tab 108. When another separator 110 is placed on the opposite (bottom) side of cathode foil 106 and is aligned with top separator 110, the laser source/beam can move along the peripheral edges of the separators 110 to complete the sealing and form a sleeve.

FIGS. 5A, 5B, and 5C illustrate another process of laser sealing. Different from the process shown in FIG. 4A, a heating element 404 may be formed on cathode tab 108 instead of a disrupted surface 402. A process window 407 represents a region where the laser welding process takes place when parameters change, and heating element 404 is shown in the middle of the process window to illustrate optimized parameters. Heating element 404 can be, for example, a meander, a simple thin rod, a wire, and/or other suitable geometry with a sufficiently small cross section, formed or bonded on the cathode tab 108. Heating element 404 can be made of any suitable conductive and resistive metal material that is able to generate heat when it conducts electric current. For example, heating element 404 can be made of one or more of nickel-chromium alloy, titanium, and other metals that form cathode tab 108. Heating element 404 is bonded onto the cathode tab 108 through any appropriate bonding means, such as an adhesive or factory bonding (e.g., welding). FIG. 5B illustrates a separator 110 positioned over a cathode tab 108, and an enlarged view of portion 405 is shown in FIG. 5C to show a laser bonding process. In this process, transfer of laser energy to the separators 110 and cathode tab 108 is increased by heating element 404, which is able to generate a desirable amount of heat during laser melting. When two separators 110 are placed on both sides of the cathode foil 106, both of the separators 110 can be simultaneously joined to the cathode tab 108 to form a sleeve.

In various embodiments, in the processes shown in FIGS. 4A, 4B, and 4C, and FIGS. 5A, 5B, and 5C, a fully transmissive layer (not shown), e.g., a lens, can be placed between the top separator 110 and the laser source 401 over the area that is intended to be joined. The fully transmissive layer can be pressed against top separator 110 to provide pressure on the materials being joined during the welding process to ensure a tighter seal and to increase the consistency of the results. The fully transmissive layer can also improve light convergence. Precision of laser bonding can be improved. In some embodiments, the fully transmissive layer is a lens having a flat bottom portion and a curved, i.e., convex or concave, top portion.

In one embodiment, the laser bonding of separators 110 can be facilitated using a non-conductive, non-metallic dye deposited on a sealing surface of at least one of the two separators to be sealed. The dye will absorb the laser energy to facilitate formation of a proper sealing layer. Accordingly, two separators 110 can be laser bonded. In one embodiment, the nonconductive dye is deposited at the peripheral edge of the bottom separator 110 (i.e., the separator furthest away from the laser). Depending on the application, the nonconductive dye can be deposited along the entire peripheral edge or only at certain locations of the peripheral edge.

As mentioned above, housing 126 of capacitor 100 is filled with a conductive electrolyte. The conductive electrolyte may be a polymer or liquid electrolyte as would be understood by a person skilled in the relevant art. Exemplary electrolytes include ethylene glycol/boric acid based electrolytes and anhydrous electrolytes based on organic solvents such as dimethylformamide (DMF), dimethylacetamide (DMA), or gamma-butyrolactone (GBL), or combinations thereof. The inventors have recognized, however, that ESR (effective series resistance) can be an issue with higher density separators, such as those described herein. Additionally, the traditional electrolyte mix with ethlylene glycol is not well suited for use with non-cellulose fiber materials. Ethylene glycol is known to bond strongly to cellulose fibers and promote wetting, but not all embodiments disclosed herein contain cellulose fibers or cellulose fibers in sufficient quantity to benefit from the bonding and wetting.

In an embodiment, the electrolyte used with capacitor 100 includes a mix of butyrolactone and ethylene glycol to maintain a low ESR in the environment of a higher density separator and lack of (or lower concentration of) cellulose fibers. A mix of butyrolactone and ethylene glycol has been shown to lower ESR of KP 60 paper (a blend of polypropylene and Kraft fibers). See U.S. Pat. No. 5,496,481, entitled “Electrolyte for Electrolytic Capacitor,” which is incorporated herein by reference in its entirety. The butyrolactone utilizes low hydrogen bonding to allow fast ion transport through the pores of the aromatic polyamide fiber material portion, and the ethylene glycol electrolyte bonds with the cellulose portion to swell the separator 110. The resistance of the separator 110 can be reduced accordingly, allowing for higher density separators, such as those described herein, to be used. The ratio of the volume of butyrolactone to the volume of ethylene glycol used may be altered as a function of the total ratio of aramid to cellulose fiber used in the separators of the capacitor, where the higher the aramid content, the higher the concentration of butyrolactone is used. The hybrid electrolyte constituent parts would wet to where they have an affinity. In various embodiments, the ratio of the volume of butyrolactone to the volume of ethylene glycol is at least about 8:2. In one embodiment, the ratio of the volume of butyrolactone to the volume of ethylene glycol is about 9:1.

In an embodiment, if the separator 110 contains an aromatic polyamide fiber material sheet but does not contain cellulose pulp or no intermediate product is formed, the conductive electrolyte contains no or little ethylene glycol. If the separator 110 contains an aromatic polyamide fiber material sheet mixed with cellulose pulp to form the intermediate product, the conductive electrolyte contains a mix of butyrolactone and ethylene glycol.

Another aspect of the present disclosure provides a method for forming separator 110.

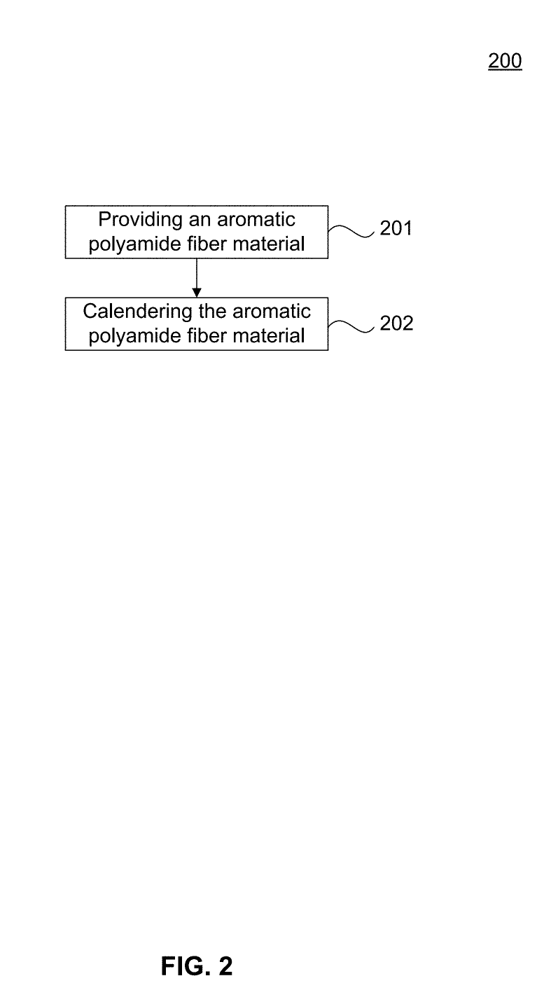

FIG. 2 illustrates an exemplary process flow 200 to form an aromatic polyamide fiber material separator.

In step S201, an aromatic polyamide fiber material is provided.

In an embodiment, non-woven aromatic polyamide fiber materials are believed by the inventors to currently be manufactured by those skilled in the art in the 30 to 40 μm thickness range with densities in the range of about 0.6 g/cm³ to about 0.8 g/cm³. Non-woven aromatic polyamide fiber materials appropriate for use in step S201 with the embodiments described herein are described in U.S. Patent Appl. Publication US2013/0288050 of U.S. patent application Ser. No. 13/871,106, entitled “Synthesis and Use of Aramid Nanofibers,” and U.S. Patent Appl. Publication US2017/0062786 of U.S. patent application Ser. No. 15/120,301, entitled “Dendrite-Suppressing Ion-Conductors from Aramid Nanofibers Withstanding Extreme Battery Conditions,” both of which are incorporated herein by reference. U.S. Patent Appl. Publication US2017/0062786 discloses a separator formed from aramid nanofibers for use as a separator in a battery. Such a separator, however, is not suitable for use in a capacitor as described herein. The capacitors described herein have voltage withstand requirements of, for example, 600 Volts, whereas batteries tend to require a voltage withstand one or even two orders of magnitude smaller.

In step S202, the aromatic polyamide fiber material is calendered. Calendering can be by any suitable hard pressure rollers capable of forming or smoothing a sheet of material. For example, the aromatic polyamide fiber material can be calendered in a paper calendering machine. In one embodiment, the aromatic polyamide fiber material is calendered in a supercalender. The calendering process applies sufficiently high pressure to decrease thickness and increase density, thereby increasing the voltage withstand of the polyamide fiber material. A person skilled in the relevant art would understand how to set calendaring parameters such as pressure, speed, and roller temperature to obtain desired characteristics of thickness, density, permeability, heat durability, and strength.

In an embodiment according to the present disclosure, the non-woven aromatic polyamide fiber material is super calendered down to about 10 μm to about 20 μm thickness and to a density of about 1.0 g/cm³ to about 1.2 g/cm³. A density above about 1.0 g/cm³ provides the necessary voltage withstand of above 600 Volts for a capacitor used at 450 Volts nominal in an ICD application, for example.

After the calendering process, the calendered aromatic polyamide fiber material can be cut or divided, e.g., through laser cutting or die punching, into a plurality of smaller sheets. The smaller sheets can be used to form separators 110 (whether as single sheets, sleeves, and/or combined with porous paper separators, as described herein).

A calendered aromatic polyamide fiber material may have a fiber diameter not greater than (i.e., less than or equal to) about 10% of the thickness of the calendered aromatic polyamide fiber material. For example, for a 20 μm-thick calendered aromatic polyamide fiber material, the fiber diameter may be less than about 2 μm, for example, about 0.1 μm to about 2 μm. In an embodiment, calendered electrospun PPTA fibers on the order of about 1 μm in diameter may be used for separators to achieve a thickness of between about 5 to about 20 μm.

In some embodiments, for an electrolytic capacitor to function at about 450 Volts, it is desired that the calendered aromatic polyamide fiber material can withstand a voltage of at least about 600 Volts. Calendered aromatic polyamide fiber material having a density of at least 1.0 g/cm³ is used to form the separators for such usages.

In an embodiment, an aromatic polyamide fiber material sheet, having a thickness of about 35 μm and a density of about −0.7 g/cm³ is provided. The aromatic polyamide fiber material sheet is placed in a supercalender and is calendered down to a calendered aromatic polyamide fiber material sheet, having a thickness of about 18 μm and a density of about 1.2 g/cm³. The calendered aromatic polyamide fiber material sheet is further cut, using a laser cutting device, into smaller sheets having shapes and sizes compatible with the separators used in an electrolytic capacitor.

In some embodiments, before calendering, a suitable pulp is added to the aromatic polyamide fiber material to form an intermediate product (i.e., aromatic polyamide fiber/pulp material). The intermediate product is further calendered or supercalendered into the calendered aromatic polyamide fiber material which has a thickness of about 10 μm to 20 μm and density of above about 1.0 g/cm³.

As described above, the pulp can contain an electrically-insulating material such as a polymeric material or a suitable natural material, such as cellulose or low lignin-containing cellulose. The specific choices of the polymeric materials and the pulp should be determined based on the desired properties of the calendered aromatic polyamide fiber material or separators. For example, different polymeric materials can be chosen to improve the strength and the heat durability of the calendered aromatic polyamide fiber material or separators. As an example, an intermediate product having an improved strength can enable the thickness of the calendered aromatic polyamide fiber material to be further reduced, further improving the packaging efficiency and energy density.

In one embodiment, the pulp is spun onto the aromatic polyamide fiber material. In another embodiment, the aromatic polyamide fiber material is soaked in the pulp to absorb the pulp. The concentration of the pulp can be adjusted such that a desired amount of the other material is absorbed into the aromatic polyamide fiber material. Depending on the types of other materials contained in the pulp, the amount of the other materials absorbed by the aromatic polyamide fiber material can be controlled such that the formed intermediate product has a desired thickness, density, thermal stability, and dimensional stability. A greater amount of pulp absorbed by the aromatic polyamide fiber material can result in a lower dimensional stability of the intermediate product. In some embodiments, the ratio of the volume of the aromatic polyamide fiber material to the volume of the pulp absorbed by the aromatic polyamide fiber material may be in the range of about 3:1 to about 1:1. In some embodiments, the ratio is about 1:1.

Optionally, a stabilizing treatment is performed to stabilizing the intermediate product to remove the solvent and to solidify the other materials absorbed in the aromatic polyamide fiber material. In various embodiments, depending on the type of the absorbed materials, the stabilizing treatment can include one or more of a drying treatment, a curing treatment, and a baking treatment. In various embodiments, other suitable treatments may also be applied to ensure the chemical stability and/or dimensional stability of the intermediate product. The specific treatment to stabilize the intermediate product should be determined according to the types and properties of the pulp and polymeric materials.

Depending on different materials, the pulp can be added into the aromatic polyamide fiber material before or after the calendering process. Also, the stabilizing treatment can be performed before or after calendering the intermediate product. That is, the specific order of forming intermediate product and stabilizing the intermediate product should be determined based on different materials used to form the intermediate product.

The intermediate product, after the stabilizing treatment, can have desired thickness, density, thermal stability, and dimensional stability to be further calendered or supercalendered into the calendered aromatic polyamide fiber material.

In an embodiment, aromatic polyamide fiber material and a cellulose-containing pulp are provided. The cellulose-containing pulp may contain a suitable weak alkaline solution. The aromatic polyamide fiber material is soaked in the cellulose-containing pulp for a desired amount of time such that a desired amount of cellulose is absorbed by the aromatic polyamide fiber material, to form an intermediate product. Further, the intermediate product is dried in a suitable stabilizing process to remove the alkaline solution such that the dried intermediate product has desired properties of chemical stability, stiffness, and dimensional stability for the calendering process and for being used as separators in an electrolytic capacitor. The dried intermediate product is further calendered or supercalendered to a calendered aromatic polyamide fiber material having a smaller thickness and a higher density, e.g., a 10 to 20 μm thickness and greater than 1 g/cm³ density. In this embodiment, the ratio of the volume of the aromatic polyamide fiber material to the volume of the cellulose-containing pulp absorbed by the aromatic polyamide fiber material is about 1:1.

In the present disclosure, term “aromatic polyamide fiber material” can represent any shape and/or form of the specified material. The specific shape and form of the material should not limit the scope of the present disclosure.

It is to be appreciated that the Detailed Description section, and not the Summary and Abstract sections, is intended to be used to interpret the claims. The Summary and Abstract sections may set forth one or more but not all exemplary embodiments of the present system and method as contemplated by the inventors, and thus, are not intended to limit the present method and system and the appended claims in any way.

Moreover, while various embodiments of the present system and method have been described above, it should be understood that they have been presented by way of example, and not limitation. It will be apparent to persons skilled in the relevant art(s) that various changes in form and detail can be made therein without departing from the spirit and scope of the present system and method. Thus, the present system and method should not be limited by any of the above described exemplary embodiments, but should be defined only in accordance with the following claims and their equivalents.

In addition, it should be understood that the figures, which highlight the functionality and advantages of the present system and method, are presented for example purposes only. Moreover, the steps indicated in the exemplary system(s) and method(s) described above may in some cases be performed in a different order than the order described, and some steps may be added, modified, or removed, without departing from the spirit and scope of the present system and method.

What is claimed is:

1. A capacitor comprising:
an anode foil;
a cathode foil; and
a separator between the cathode foil and the anode foil, wherein the separator comprises an aromatic polyamide fiber material having a density of greater than about 1.0 g/cm³, wherein the separator has a thickness in a range of about 5 μm to about 20 μm, and wherein the separator is able to withstand a voltage of at least 460 Volts.

2. The capacitor of claim 1, further comprising a conductive electrolyte, wherein the conductive electrolyte fills between the cathode foil and the anode foil and contains butyrolactone.

3. The capacitor of claim 1, wherein the separator has a density of between greater than 1.0 g/cm³ and about 1.2 g/cm³.

4. The capacitor of claim 1, wherein the separator further comprises a first porous paper material calendered to the aromatic polyamide fiber material, wherein the first porous paper material has a thickness in a range of about 5 μm to about 10 μm and the aromatic polyamide fiber material has a thickness in a range of about 5 μm to about 10 μm.

5. The capacitor of claim 1, further comprising a second separator comprising a second porous paper, wherein:
the first porous paper and the second porous paper are arranged over both surfaces of the cathode foil to form a first sleeve such that the cathode foil is arranged in the first sleeve; and
the aromatic polyamide fiber material is arranged between the first sleeve and the anode foil.

6. The capacitor of claim 5, wherein the second separator further comprises a second aromatic polyamide fiber material, wherein the aromatic polyamide fiber material of the first separator and the second aromatic polyamide fiber material of the second separator form a second sleeve enclosing the first sleeve, wherein the second sleeve enclosing the first sleeve is bonded together with a laser seal in an area outside the perimeter of the first sleeve.

7. The capacitor of claim 5, wherein the second separator further comprises a second aromatic polyamide fiber material, wherein the aromatic polyamide fiber material of the first separator and the second aromatic polyamide fiber material of the second separator form a second sleeve enclosing the first sleeve, wherein the second sleeve enclosing the first sleeve is bonded on a cathode tab connected to the cathode foil with a laser seal.

8. The capacitor of claim 1, wherein the separator further comprises a porous paper material mixed with the aromatic polyamide fiber material to form a composite, wherein the composite has a density of greater than about 1.0 g/cm³, a thickness in the range of about 5 μm to about 20 μm, and wherein the composite is able to withstand a voltage of at least 600 Volts.

9. The capacitor of claim 7, wherein the cathode tab comprises texturing or macrostructures.

10. The capacitor of claim 7, wherein the cathode tab comprises a heating element configured to increase transfer of laser energy to the second sleeve and the cathode tab.

11. The capacitor of claim 2, wherein the separator comprises cellulose and the conductive electrolyte further contains ethylene glycol, the ratio of the volume of butyrolactone to the volume of ethylene glycol being about at least 8:2.

12. A method for fabricating a capacitor of an implantable cardioverter defibrillator (ICD) or a subcutaneous implantable cardioverter defibrillator (SICD), the method comprising:
calendering an aromatic polyamide fiber material down to a thickness of about 5 μm to about 20 μm and to a density of about 1.0 g/cm³ to about 1.2 g/cm³.

13. The method of claim 12, further comprising: before calendaring the aromatic polyamide fiber material, adding a pulp into the aromatic polyamide fiber material such that the pulp is combined with the aromatic polyamide fiber material for forming the calendered fiber material.

14. The method of claim 13, wherein a ratio of a volume of the aromatic polyamide fiber material to a volume of the pulp absorbed by the aromatic polyamide fiber material is in a range of about 3:1 to about 1:1.

15. The method of claim 12, further comprising: before calendaring the aromatic polyamide fiber material, stacking the aromatic polyamide fiber material with a porous paper sheet, and wherein the aromatic polyamide fiber material and the porous paper sheet are calendered together to form a double-layered separator, wherein the thickness of the composite double-layered separator is about 20 μm or less.

16. The method of claim 12, further comprising bonding the aromatic polyamide fiber material to an electrode tab of the capacitor using a laser weld.

17. The method of claim 16, further comprising providing a disrupted surface on an electrode tab for the aromatic polyamide fiber material to move into when the material is heated to or near its melting point with a laser during welding.

18. The method of claim 16, further comprising forming a heating element on a cathode tab.

19. The method of claim 12, further comprising placing cathode foil between a top and a bottom separator comprising the calendered aromatic polyamide fiber material;
placing a transmissive layer between the top separator and a laser source over an area to be joined, wherein the transmissive layer is configured to improve light convergence to improve the precision of the laser bonding; and
simultaneously joining the cathode tab with the first and second separators to form a separator sleeve.

20. The method of claim 19, further comprising pressing the transmissive layer against the top separator, so as to provide pressure on the area to be joined.
